(19) United States
(12) Patent Application Publication
(10) Pub. No.: US 2025/0375427 A1
(43) Pub. Date: Dec. 11, 2025

(54) METHODS OF TREATING GLIOBLASTOMA WITH PRODRUGS OF RILUZOLE

(71) Applicant: Biohaven Therapeutics Ltd., New Haven, CT (US)

(72) Inventors: Irfan QURESHI, Hackensack, NJ (US); Alissa RYBICKI, Deep River, CT (US); Katheryn GROSSMAN, New Haven, CT (US); Mary K. DONOHUE, Wallingford, CT (US)

(21) Appl. No.: 18/876,651

(22) PCT Filed: Jun. 26, 2023
(86) PCT No.: PCT/US2023/069038

§ 371 Date: Dec. 18, 2024

Related U.S. Application Data
(60) Provisional application No. 63/355,631, filed on Jun. 26, 2022.

(51) Int. Cl. A61K 31/428; A61K 31/175; A61K 31/495; A61K 38/06

(52) U.S. Cl. CPC A61K 31/428; A61K 31/175; A61K 31/495; A61K 38/06

(57) ABSTRACT

Provided is a method of treating glioblastoma in a patient in need of such treatment, comprising administering to the patient a first dosage form comprising a therapeutically active amount of troriluzole or a pharmaceutically acceptable salt thereof.

### FIELD OF THE INVENTION

The present invention relates to treatment of cancer with prodrugs of riluzole. In particular, the present invention relates to treatment of glioblastoma with troriluzole as a monotherapy or in combination with another anti-cancer agents and/or ionizing radiation.

### BACKGROUND OF THE INVENTION

Glioblastoma (GBM) is a fast-growing and aggressive brain tumor that invades the nearby brain tissue, but generally does not spread to distant organs. In adults, GBM occurs most often in the cerebral hemispheres, especially in the frontal and temporal lobes of the brain. GBM is a devastating brain cancer that has no cure and can result in death in six months or less. Thus, new medicines are urgently needed to treat this aggressive type of cancer.

Troriluzole is a novel tripeptide prodrug of the glutamate modulating agent riluzole. Riluzole is the active ingredient in the reference listed drug, RILUTEK®, which is approved for the treatment of amyotrophic lateral sclerosis (ALS). In addition, clinical and translational research studies suggest that riluzole may also be an effective treatment for a number of other indications. However, the optimal use of riluzole is limited by several factors. Riluzole tablets have approximately 60% bioavailability, attributed to high first-pass effects in the liver and to variable metabolism by the heterogeneously expressed CYP1A2 enzyme. These factors lead to a relatively high degree of pharmacokinetic (PK) variability after administration of riluzole. Riluzole is also associated with reduced absorption when it is taken with meals (i.e., a negative food effect), which can lead to low systemic exposures. Therefore, it is recommended that riluzole be administered one hour before or two hours after a meal (i.e., a 3 hour fast). Riluzole has dose-dependent effects on liver function tests (LFTs). The riluzole drug substance itself has other intrinsic limitations including very low solubility in water, poor oral palatability, pH-dependent chemical stability, and intense oral numbness if administered directly to the oral mucosa.

Troriluzole was developed to mitigate the aforementioned limitations of riluzole. Troriluzole is a novel, rationally designed third generation tripeptide prodrug of riluzole that potentially offers improved bioavailability, pharmacology, safety, and dosing. Troriluzole is actively absorbed in the gut (via the peptide transporter [PepT1]), is not subject to a negative food effect, is rapidly cleaved by aminopeptidases in the systemic circulation to release the active metabolite, is expected to generate predictable exposures of its active metabolite, bypasses first-pass metabolism, reduces riluzole burden on the liver, and allows exploration of higher concentrations of active metabolite.

It has been reported that troriluzole may be useful to treat certain types of cancer. However, no evidence has been presented for troriluzole to treat glioblastoma.

### SUMMARY OF THE INVENTION

The present invention is directed to methods of treating glioblastoma by administering a prodrug of riluzole, as either a monotherapy or a combination therapy with another anti-cancer agent and/or ionizing radiation.

In an embodiment, a method of treating glioblastoma in a patient in need of such treatment is provided. The method includes administering to the patient a first dosage form including a therapeutically active amount of troriluzole or a pharmaceutically acceptable salt thereof.

In another embodiment, the method may further include administering to the patient a second dosage form including a therapeutically active amount of temozolomide or a pharmaceutically acceptable salt thereof.

In still another embodiment, the method may further include administering to the patient a third dosage form including a therapeutically active amount of lomustine or a pharmaceutically acceptable salt thereof.

In yet another embodiment, the method may further include irradiating glioblastoma cells with a therapeutically effective dose of ionizing radiation.

### BRIEF DESCRIPTION OF THE DRAWINGS

These and/or other aspects will become apparent and more readily appreciated from the following description of the embodiments, taken in conjunction with the accompanying drawings in which FIGURE shows mechanism of action of troriluzole.

### DETAILED DESCRIPTION OF THE INVENTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

Additional aspects will be set forth in part in the description which follows and, in part, will be apparent from the description.

In an embodiment, provided is a method of treating glioblastoma in a patient in need of such treatment. The method includes administering to the patient a first dosage form comprising a therapeutically active amount of troriluzole or a pharmaceutically acceptable salt thereof.

Troriluzole is a compound having the following chemical formula:

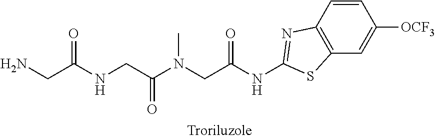 [SMILES: CN(CC(=O)Nc1nc2ccc(OC(F)(F)F)cc2s1)C(=O)CNC(=O)CN]

The first dosage form may include 10 to 500 mg of troriluzole or an equivalent amount of a pharmaceutically acceptable salt thereof. In some aspects, the first dosage form may include 10, 20, 30, 40, 50, 60, 70, 80, 90, 100, 150, 200, 250, 300, 350, 400, 450, or 500 mg of troriluzole or an equivalent amount of a pharmaceutically acceptable salt thereof. For example, the first dosage form may include 100 mg or 200 mg of troriluzole or an equivalent amount of a pharmaceutically acceptable salt thereof.

The first dosage form may be administered once a day, twice a day, three times a day, four times a day, or with any other frequency, as needed. For example, the first dosage form may be administered once a day or twice a day.

The duration of treatment can be from several days to several weeks to several month. For example, the treatment may be administered for one, two, three, four, five, six, or seven days. In another example, the treatment may be administered for one, two, three, four, five, six, or seven weeks. In another example, the treatment may be administered for one, two, three, four, five, six, or seven months. In an embodiment, the treatment may be administered for one, two, three, four, five, six, seven, eight, nine, ten, 11, 12, 13, 14, 15, 16, 17, 18, 19, 20, 21, 22, 23, 24, 25, 26, 27, or 28 days. In another example, the treatment may be administered for a cycle consisting of 28 days. In another example, the treatment may be administered for one, two, three, four, five, six, seven, eight, nine or ten 28-day cycles.

In an aspect, the duration of treatment may be four weeks, and troriluzole or a pharmaceutically acceptable salt thereof may be administered in an amount of 100 mg twice a day for the first two weeks and in amount of 200 mg twice a day for the other two weeks.

In an aspect, the total amount of troriluzole or a pharmaceutically acceptable salt thereof administered to the patient during the day may be 50 to 1000 mg. For example, the total amount of troriluzole administered to the patient during the day may be 50, 100, 150, 200, 250, 300, 350, 400, 450, 500, 550, 600, 650, 700, 750, 800, 850, 950, or 1000 mg. For example, the total amount of troriluzole administered to the patient during the day may be 200 mg, 300 mg, or 400 mg.

In another embodiment, troriluzole may be paired with temozolomide to treat glioblastoma. Temozolomide is a medication to treat brain tumors that has the following chemical structure:

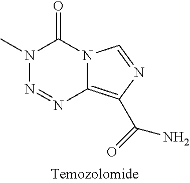

Troriluzole and temozolomide may be included in the same or different dosage forms. In an aspect, the method of treating glioblastoma with troriluzole may further include administering to the patient a second dosage form including a therapeutically active amount of temozolomide or a pharmaceutically acceptable salt thereof. The second dosage form may include 10 to 300 mg of temozolomide or an equivalent amount of a pharmaceutically acceptable salt thereof. For example, the second dosage form may include 10, 15, 20, 25, 30, 35, 40, 45, 50, 55, 60, 65, 70, 75, 80, 85, 90, 95, 100, 125, 150, 175, 200, 225, 250, 275, or 300 mg of temozolomide or an equivalent amount of a pharmaceutically acceptable salt thereof. For example, the second dosage form may include 75 or 150 mg of temozolomide or an equivalent amount of a pharmaceutically acceptable salt thereof.

The second dosage form may be administered once a day, twice a day, three times a day, or four times a day. For example, the second dosage form may be administered once a day or twice a day.

In an aspect, the total amount of temozolomide or a pharmaceutically acceptable salt thereof administered to the patient during the day may be 50 to 300 mg. For example, the total amount of temozolomide or a pharmaceutically acceptable salt thereof administered to the patient during the day may be 75 mg. In another example, the total amount of temozolomide or a pharmaceutically acceptable salt thereof administered to the patient during the day may be 150 mg.

The glioblastoma may be newly diagnosed O⁶-methylguanine-DNA-methyltransferase (MGMT) methylated glioblastoma or O⁶-methylguanine-DNA-methyltransferase (MGMT) unmethylated glioblastoma.

In another embodiment, troriluzole may be paired with lomustine to treat glioblastoma. Lomustine is an alkylating nitrosourea compound used in chemotherapy that has the following structure:

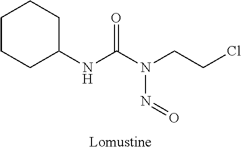 [SMILES: O=NN(CCCl)C(=O)NC1CCCCC1]

Troriluzole and lomustine may be included in the same or different dosage forms. In an aspect, the method for treating glioblastoma with troriluzole may further include administering to the patient a third dosage form including a therapeutically active amount of lomustine or an equivalent amount of a pharmaceutically acceptable salt thereof.

In an aspect, the third dosage form may include 10 to 200 mg of temozolomide or an equivalent amount of a pharmaceutically acceptable salt thereof. For example, the second dosage form may include 10, 15, 20, 25, 30, 35, 40, 45, 50, 55, 60, 65, 70, 75, 80, 85, 90, 95, 100, 125, 150, 175, or 200 mg of temozolomide or an equivalent amount of a pharmaceutically acceptable salt thereof. In an aspect, the third dosage form may include 50 mg of lomustine or an equivalent amount of a pharmaceutically acceptable salt thereof. In another aspect, the third dosage form may include 100 mg of lomustine or an equivalent amount of a pharmaceutically acceptable salt thereof.

The third dosage form may be administered once a day, twice a day, three times a day, or four times a day. For example, the third dosage form may be administered once a day.

The total amount of lomustine or a pharmaceutically acceptable salt thereof administered to the patient during the day may be 50 to 200 mg. For example, the total amount of lomustine or a pharmaceutically acceptable salt thereof administered to the patient during the day may be 100 mg. The glioblastoma may be recurring glioblastoma.

In another embodiment, troriluzole may be paired with an additional anti-cancer agent to treat glioblastoma. The additional anti-cancer agent may include regorafenib, paxalisib, VAL-083, VT1021, or any combination thereof.

In an embodiment, troriluzole may be paired with regorafenib to treat glioblastoma. Regorafenib is an oral multi-kinase inhibitor which targets angiogenic, stromal and oncogenic receptor tyrosine kinase (RTK). Regorafenib has the following chemical structure:

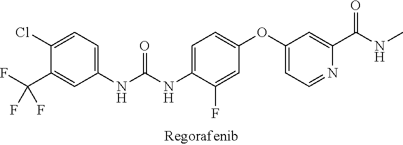 [SMILES: CNC(=O)c1cc(Oc2ccc(NC(=O)Nc3ccc(Cl)c(C(F)(F)F)c3)c(F)c2)ccn1]

Troriluzole and regorafenib may be included in the same or different dosage forms. In an aspect, the method of treating glioblastoma with troriluzole in a patient in need of such treatment further includes administering to the patient a fourth dosage form comprising a therapeutically active amount of regorafenib or a pharmaceutically acceptable salt thereof.

In another embodiment, troriluzole may be paired with paxalisib to treat glioblastoma. Paxalisib is a phosphatidylinositol 3-kinase (PI3K) inhibitor with potential antineoplastic activity that has the following chemical structure:

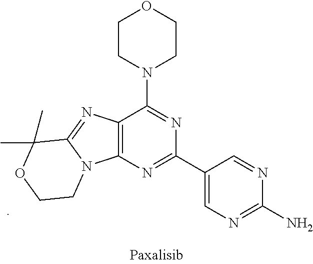 [SMILES: CC1(C)OCCn2c1nc1c(N3CCOCC3)nc(-c3cnc(N)nc3)nc12]

Troriluzole and paxalisib may be included in the same or different dosage forms. In an aspect, the method of treating glioblastoma with troriluzole in a patient in need of such treatment further includes administering to the patient a fifth dosage form comprising a therapeutically active amount of paxalisib or a pharmaceutically acceptable salt thereof.

In another embodiment, troriluzole may be paired with VAL-083 to treat glioblastoma. VAL-083 is a bifunctional DNA-targeting agent which preclinically demonstrated activity against multiple solid and hematologic tumors. VAL-083 has the following chemical structure:

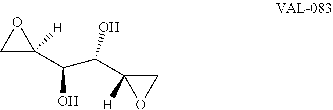

Troriluzole and VAL-083 may be included in the same or different dosage forms. In an aspect, the method of treating glioblastoma with troriluzole in a patient in need of such treatment further includes administering to the patient a sixth dosage form comprising a therapeutically active amount of VAL-083 or a pharmaceutically acceptable salt thereof.

In another embodiment, troriluzole may be paired with VT1021 to treat glioblastoma. VT1021 is a dual modulating compound developed by Vigeo Therapeutics Ltd. that blocks the CD47 immune checkpoint and activates CD36, which induces apoptosis and increases the M1:M2 macrophage ratio.

Troriluzole and VT1021 may be included in the same or different dosage forms. In an aspect, the method of treating glioblastoma with troriluzole in a patient in need of such treatment further includes administering to the patient a seventh dosage form comprising a therapeutically active amount of VT1021 or a pharmaceutically acceptable salt thereof.

In another embodiment, the method for treating glioblastoma may further include irradiating glioblastoma cells with a therapeutically effective dose of ionizing radiation.

In another embodiment, provided a pharmaceutical composition including troriluzole and additional anti-cancer agent selected from the group consisting of temozolomide, lomustine, regorafenib, paxalisib, VAL-083, VT1021, and any combination thereof in an amount effective to treat glioblastoma. For example, the pharmaceutical composition may include troriluzole and temozolomide or a pharmaceutically acceptable salt thereof. In another example, the pharmaceutical composition may include troriluzole and lomustine or a pharmaceutically acceptable salt thereof. In another example, pharmaceutical composition may include troriluzole and regorafenib or a pharmaceutically acceptable salt thereof. In another example, the pharmaceutical composition may include troriluzole and paxalisib or a pharmaceutically acceptable salt thereof. In another example, the pharmaceutical composition may include troriluzole and VAL-083 or a pharmaceutically acceptable salt thereof. In another example, the pharmaceutical composition may include troriluzole and VT1021 or a pharmaceutically acceptable salt thereof.

### Method of Treatment Rationale

A new drug, troriluzole, is being developed for the treatment of neurologic and psychiatric disorders, as well as other potential indications. Troriluzole is a novel, orally administered prodrug of the glutamatergic agent riluzole. The FDA originally approved riluzole (RILUTEK®) for the treatment of patients with Amyotrophic Lateral Sclerosis (ALS)¹. Riluzole is only indicated for ALS and has a number of non-desirable attributes that have limited its clinical development and use. Troriluzole is a tripeptide prodrug of the glutamate modulating agent riluzole that has been optimized for improved bioavailability, pharmacokinetics and dosing.

The proposed study in glioblastoma multiforme (GBM) is based on accumulating evidence that implicates glutamatergic deregulation in the pathophysiology of a number of different tumors. In addition, preclinical and clinical evidence with troriluzole (or its active metabolite, riluzole), suggests that troriluzole has the potential to be an effective treatment for these tumors that is complementary to existing standard of care therapies (SOC). The high unmet need, together with the available data, provide a compelling rationale for the development of troriluzole as a supplemental treatment for GBM.

Riluzole was originally developed as a centrally acting muscle relaxant and subsequently as an anticonvulsant and neuroprotective agent. The primary mode of action of riluzole is reducing synaptic levels of glutamate. This occurs via two mechanisms: (1) riluzole increases glutamate uptake from the synapse by glial cells and (2) riluzole decreases glutamate release from presynaptic neurons. Riluzole decreases the release of glutamate from presynaptic neurons because it inactivates voltage-gated sodium channels on glutamatergic nerve terminals that mediate presynaptic neuronal depolarization. In addition, riluzole increases glutamate uptake from the synapse, by augmenting the expression and function of excitatory amino acid transporters (i.e., EAAT2) located on glial cells that play a key role in clearing glutamate from the synapse²⁻⁸. Moreover, riluzole has a range of other therapeutically relevant pharmacological effects that include the modulation of neurotransmitter signaling and ion channel function, and enhancement of neurotrophic and pro-survival cellular signaling pathways.

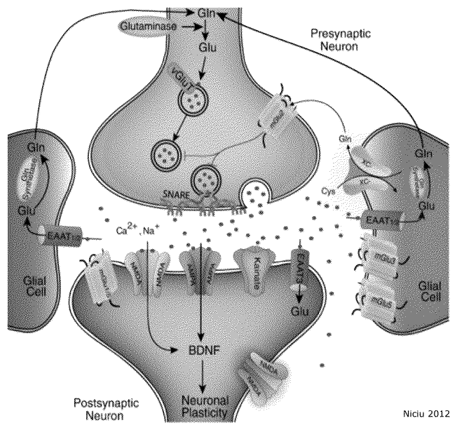

As shown in FIG. 1, troriluzole reduces extracellular glutamate levels by: (1) facilitating extracellular glutamate reuptake by increasing expression and function of excitatory amino acid transporters on glial cells, and (2) reducing presynaptic glutamate release by inhibiting voltage-gated sodium channels.

The therapeutic agents included in GBM AGILE are selected through a rigorous scientific evaluation process. For more information, please see Reference 9.

Glutamate promotes GBM maintenance, proliferation, migration, and invasion. GBM is characterized by alterations in cellular metabolism, especially glutamate homeostasis. Glutamate transporter dysregulation (SXC [CI-dependent cystine/glutamate exchanger], EAAT) leads to excess secretion into the microenvironment and decreased glutamate reuptake and excess glutamate acts in an autocrine/paracrine manner to activate glutamate receptors and promote tumor growth and invasion. Excess glutamate also has inhibitory effects on T cell function and leads to excitotoxic neuronal death. Tumor growth/invasion and excitotoxicity lead to GBM-associated seizures. Neuronal activity may stimulate tumor progression via glutamatergic neuronal-gliomal synaptic communication. Non-clinical evidence supports studying anti-glutamatergic agents as potential GBM therapeutics.

Troriluzole was selected to be included in the trial because of its novel targeted therapy as a potent glutamate modulator that can be a potential treatment for glioblastoma, showing minimal toxicity.

Troriluzole acts by reducing extracellular glutamate levels at synaptic junctions. This is done by two mechanisms: (1) facilitating extracellular glutamate reuptake by increasing the expression and function of excitatory amino acid transporters (EAATs) on glial cells, and (2) reducing presynaptic glutamate release by inhibiting voltage-gated sodium channels.

Aside from its mechanism of action, there are other prima facie reasons to believe that troriluzole may provide benefit to patients with GBM. Preclinical data and clinical data have shown valuable therapeutic effects.

Preclinical data in GBM models showed the following important conclusions about riluzole and troriluzole:
Riluzole combined with radiation enhances radiation-induced cell cytotoxicity in U87MG cell line in anchorage independent colony formation assay¹⁰.
Riluzole combined with radiation enhances sensitivity to radiation in CAM assay in 64SP stem like cells¹¹.
Riluzole combined with temozolomide (TMZ) enhances the effects of TMZ via suppression of O⁶-methylguanine-DNA-methyltransferase (MGMT) expression¹².
Troriluzole treatment induced a survival benefit, promoted proliferation of tumor-infiltrating T cells, and synergized with anti-PD-1 immunotherapy to further improve survival and reduce T regulatory cells in the glioma tumor microenvironment (TME)¹³.
Preclinical IF data suggest that there was increased concentration of CD4+ T cells in TME after troriluzole treatment. Furthermore, troriluzole monotherapy and combination with anti-PD-1 therapy led to increased CD3+ tumor infiltrating lymphocyte proliferation¹³.

Preliminary clinical data from trials of troriluzole (or its active metabolite) also suggest potential for pharmacodynamic and clinical response in non-GBM tumors.

In summary, glutamatergic signaling has been shown to promote proliferation, migration, and invasion in glioblastoma. More recently the interaction between neuronal signaling and glioma cells has created additional interest in targeting glutamate receptors. Troriluzole is a glutamate modulator and in animal models has been shown to impact glioblastoma growth. It has an acceptable safety profile and has evidence for blood brain barrier penetration. Combination studies with riluzole in glioma model systems has shown enhanced sensitivity to both radiation and TMZ. For these reasons, the arm selection committee feels that incorporating troriluzole into the GBM AGILE trial provides the opportunity to more fully evaluate its efficacy in GBM.

### Troriluzole Dose Selection Rationale

The study will start by assessing troriluzole administered 200 mg BID in the dose finding phase and dose confirmation will be performed. This dosage was selected based on cumulative experience, including nonclinical toxicology and safety/tolerability, pharmacokinetic, and pharmacodynamic data. Importantly, the MTD of troriluzole was determined to be 140 mg QAM+280 mg PO QPM. The 200 mg BID dosage represents a simpler regimen for patients to follow (the same dose is administered approximately 12 hours apart) without an appreciable impact on exposures to riluzole. A population PK model utilizing data from healthy subjects, as well as patients across several indications being studied with troriluzole (SCA, OCD, AD, and GAD) was developed and used to simulate riluzole exposures following administration of troriluzole 200 mg BID and the MTD of 140 mg QAM+280 mg QPM. Simulated riluzole Cₘₐₓ and AUC₀₋₂₄ in both healthy subjects and patients are presented in Table 1.

TABLE 1
Median (90% prediction interval) steady state riluzole exposure
parameters following administration of troriluzole^{a}

| | Riluzole AUC₀₋₂₄ (ng*h/mL) | | Riluzole Cₘₐₓ (ng/mL) | |
|---|---|---|---|---|
| Troriluzole | Healthy | | Healthy | |
| Dose | Subjects | Patients | Subjects | Patients |
| 200 mg BID | 3070 | 3100 | 233 | 226 |
| | (1900, 5780) | (1020, 7290) | (131, 423) | (94, 576) |
| 140 mg QAM + | 3420 | 3450 | 276 | 260 |
| 200 mg QPM | (1970, 6830) | (1080, 10900) | (148, 491) | (115, 664) |

^{a}The simulated population was assumed to be 50% female, with age and weight randomly sampled with replacement by sex from the dataset. The simulations assumed no fluvoxamine use, and assumed the evening dose was administered in the fed state. The fed status for morning dose was assumed to be 50%.

As demonstrated in Table 1, simulated riluzole exposures following administration of troriluzole 200 mg BID are similar to those following administration of the MTD and are anticipated to be safe in the glioblastoma population. A dose finding phase will be implemented to confirm this recommended dose of troriluzole in combination with radiotherapy and temozolomide, and lomustine, and to ensure the safety of the patients.

### Overview of Troriluzole Arm

The troriluzole arm is open to patients with 1) NDU GBM, 2) NDM GBM, and 3) recurrent GBM of any methylation status. Troriluzole is being studied in combination with temozolomide and radiation in newly diagnosed patients, as well as in combination with lomustine in recurrent disease patients for the first time, and hence, a dose finding phase is being implemented to confirm the dose of troriluzole to ensure the safety of the combinations in glioblastoma patients. A rolling 6 design is being carried out to determine the starting doses.

Once a patient is assigned to the troriluzole experimental arm, the patient will undergo supplemental safety assessments including biweekly collection of adverse events, dose modification profiles, hematology, serum chemistry panels and coagulation profiles. These additional safety assessments, referred to as Enhanced Safety Management (ESM), will be conducted in approximately the initial 30 patients assigned to the troriluzole experimental arm: 20 newly diagnosed patients (NDU and NDM) and 10 recurrent patients.

The troriluzole arm as part of GBM AGILE will be comprised of 2 stages: the first will be a Bayesian adaptively randomized screening stage (Stage 1) to identify the effectiveness of troriluzole in improving survival. In Stage 1, a target maximum of 200 patients will be randomized to troriluzole across all applicable disease subtypes. The second stage (Stage 2) will use a fixed randomization to confirm efficacy signals identified in Stage 1 and to support drug approval. The target maximum sample size in Stage 2 is 50 patients in the experimental arm. The initial screening stage (Stage 1) uses a Bayesian adaptive randomization algorithm to allocate patients to experimental or control arms based on their disease subtype (defined below).

### Dose Finding Phase

Troriluzole has been studied in various diseases and oncology spaces and has been well tolerated. However, GBM AGILE is the first trial where troriluzole is being studied in combination with temozolomide and radiation in newly diagnosed patients and in combination with lomustine in recurrent disease patients. In order to confirm the recommended dose of troriluzole in combination with radiotherapy and temozolomide, and lomustine, and ensure the safety of the patients, troriluzole will go through a dose finding phase. This phase of the study will be at select sites in the United States only, and will be completed prior to activating the arm in additional regions.

Newly Diagnosed Patients. The first 6 patients in the newly diagnosed subtype will receive troriluzole 100 mg BID for the first two weeks followed by 200 mg BID for the next two weeks in combination with temozolomide and radiation therapy. If no DLTs or 1 DLT is observed in these 6 patients (1/6) these patients will continue in the study and will continue to undergo Enhanced Safety Monitoring (ESM) (described in detail below) and this dose will be determined as dose level 0 or the starting dose.

If there are 2 DLTs in the first 6 patients (2/6), then the dose will be de-escalated to 100 mg once every morning (qAM) and 200 mg once every evening (qPM) for 4 weeks. 6 patients will then be receiving 100 mg qAM followed by 200 mg qPM for 4 weeks and observed for DLTs. If no DLTs or 1 DLT is observed in these 6 patients (1/6) these patients will continue in the study and will continue to undergo ESM (described in detail below) and this dose of 100 mg qAM and 200 mg qPM will be confirmed as dose level 0 or starting dose.

If there are 2 DLTs in these 6 patients (2/6), then the dose will be de-escalated to 100 mg BID. 6 patients will then be receiving 100 mg BID for 4 weeks and observed for DLTs. If no DLTs or 1 DLT is observed in 6 patients (1/6) these patients will continue in the study and will continue to undergo ESM (described in detail below) and this dose of 100 mg BID will be confirmed as dose level 0 or starting dose.

Recurring Disease Patients. Similarly, the first 6 patients in the recurrent disease subtype will receive troriluzole at 100 mg BID for the first two weeks followed by 200 mg BID for the next two weeks in combination with lomustine. These patients will be monitored for DLTs for 4 weeks. If no DLTs or 1 DLT is observed in 6 patients (1/6) these patients will continue in the study and will continue to undergo ESM (described in detail below) and 200 mg BID dose of troriluzole will be determined as dose level 0 or the starting dose.

If there are more than 2/6 DLTs in the first 6 patients, then the dose will be de-escalated to 100 mg once every morning (qAM) and 200 mg once every evening (qPM) for 4 weeks. 6 patients will then be receiving 100 mg qAM followed by 200 mg qPM for 4 weeks and observed for DLTs. If no DLTs or 1 DLT is observed in 6 patients (1/6) these patients will continue in the study and will continue to undergo ESM (described in detail below) and this dose of 100 mg qAM and 200 mg qPM will be confirmed as dose level 0 or starting dose.

If there are 2 DLTs in these 6 patients (2/6), then the dose will be de-escalated to 100 mg BID. 6 patients will then be receiving 100 mg BID for 4 weeks and observed for DLTs. If no DLTs or 1 DLT is observed in 6 patients (1/6) these patients will continue in the study and will continue to undergo ESM (described in detail below) and this dose of 100 mg BID will be confirmed as dose level 0 or starting dose.

If there is a dose de-escalation, patients that received a higher dose during the dose finding phase and did not have any DLTs can continue on the trial and receive the confirmed starting dose. However, these patients will not be included in the final analysis.

The DLTs should be reversible and monitorable within 4 weeks. Evaluable DLTs will be considered as those where the patients have received 75% or more of the study drug within the dose finding phase. If a patient has a DLT prior to receiving 75% of the study drug during this phase, then a new patient will be added to the rolling 6 design to replace this patient in order to complete the evaluation. During the dose finding phase, patients will be assessed biweekly for AEs. In addition, patients will also undergo the ESM evaluations during this phase.

After the 6th patient is enrolled in the dose finding phase, as per the rolling 6 design, enrollment to the troriluzole arm will pause for 4 weeks to allow for DLT evaluations in this patient. Upon completion of the DLT evaluations, the starting dose will be confirmed and enrollment on the troriluzole arm for both the newly diagnosed and recurrent subtypes will resume.

### Enhanced Safety Management (ESM)

Enhanced safety management is a seamless process for new arms with different classes of combined agents for which there is limited safety data to supplement and support their inclusion in the GBM AGILE trial. The decision to employ ESM for these arms to enter GBM AGILE is based on the recommendations of the Arm Selection Committee. ESM provides an opportunity for agents with a well-tolerated safety profile to be tested in all subgroups (newly diagnosed methylated, newly diagnosed unmethylated and recurrent patients) in GBM AGILE. ESM will be at select sites in the United States only, and will be completed prior to activating the arm in additional regions.

Once a patient is assigned to the troriluzole experimental arm, the patient will undergo supplemental safety assessments including biweekly collection of adverse events, dose modification profiles, hematology, serum chemistry panels and coagulation profiles. These additional safety assessments will be conducted in approximately the initial 30 patients assigned to the troriluzole experimental arm. While there is no minimum or maximum number of patients, it is estimated to be approximately 20 newly diagnosed patients and 10 recurrent patients.

These additional assessments will be entered into a monthly report that will be reviewed by Global Principal Investigator (PI) and medical monitor (MM) for submission to 1-2 clinical members of the Data and Safety Monitoring Board (DSMB).

ESM will be monitored by the clinical leadership and the DSMB in the following way:
Real Time: Dose modifications or temporary drug hold based on AE assessments will be monitored in real time by Global PI and MM.
Monthly Report: The Global PI and MM will review the monthly data and will look to 1 to 2 clinical members of the DSMB to recommend continuing or suspending the ESM data collection or stop the investigational arm.
Quarterly Report: Comprehensive ESM data till date will be presented at the quarterly DSMB meetings. All members of DSMB will review the decisions made from the monthly reports. If the decision to suspend the ESM data collection happens between the quarterly DSM meetings, an ad-hoc review by all the DSMB members may be requested.

If and when there is a decision to stop the ESM by the DSMB, the investigator will be notified and the additional safety measurements are no longer required.

If there is a Grade 5 event or any two Grade 4 events considered at least possibly related to troriluzole by the DSMB during the ESM period, accrual of patients will be suspended until a thorough safety review is conducted by the DSMB prior to resuming enrollment as advised by members of the DSMB.

### Treatment Procedures

Required Tests or Assessments. The following procedures will be performed for newly diagnosed and recurrent patients at each visit prior to administration of troriluzole (+3 days), except when noted otherwise below.
Neurologic examination, a clinical assessment of a neurologic function, performed per local practice. NOTE: For recurrent patients, if performed within 14 days of randomization AND Cycle (C) 1 Day (D) 1, then the patient does not require the additional assessment at C1D1.
Vital signs (including BP, temperature, and heart rate) performed per local practice.
ECG should be collected performed during the screening period and as follows:
   For newly diagnosed patients: Day 1 and Day 29 of the treatment period and Day 1 of every cycle during the maintenance period
   For recurrent patients: Day 1 of every cycle
   Additional ECG monitoring should be performed as clinically indicated for all participants on the troriluzole arm
Karnofsky performance status performed per local practice (see Table 2 below).
Adverse event assessment.
Concomitant medications.
Corticosteroid use.
QOL assessments (EORTC QLQ-C-30, EORTC QLQ-BN20, and EQ-5D-5L) should be performed prior to study treatment on C1D1, then prior to patient receiving any daily results (if feasible), and coincide with imaging response assessments as follows:
   For newly diagnosed patients: every 8 weeks (+4 weeks).
   For recurrent patients: every 6 weeks (+1 week).
Laboratory blood or urine tests (collected and assessed locally):
   Hematology: Complete blood count with differential (absolute), including but not limited to:
      Red blood cell count.
      Hemoglobin.
      Hematocrit.
      White blood cell count.
      Absolute neutrophil count.
      Absolute lymphocyte count.
      Platelet count.
   Serum Chemistry: Chemistry panel including, but not limited to:
      Sodium.
      Potassium.
      Calcium.
      Chloride.
      Blood urea nitrogen.
      Creatinine.
      Fasting Glucose.
      Total Bilirubin (if total bilirubin is greater than the upper limit of normal, direct and indirect bilirubin should be performed).
      Aspartate aminotransferase/serum glutamic oxaloacetic transaminase (AST/SGOT).
      Alanine aminotransferase (ALT/SGPT).
      Lipase.
      Amylase.
   Urinalysis, per local practice.
Administration or dispensation of study treatment.
MRI for response assessment/tumor evaluation to be conducted:
   For newly diagnosed patients: every 8 weeks (±4 weeks).
   For recurrent patients: every 6 weeks (±1 week).
      NOTE: For recurrent patients, if the MRI scan is performed within 14 days of randomization AND C1D1, then the patient does not require the additional MRI at C1D1. MRI scans should be performed per the frequency described above for newly diagnosed and recurrent patients, regardless of treatment delays. If the criteria for “preliminary” progressive disease is met, a confirmatory MRI scan must be performed no sooner than 4 weeks later per Section 14.2.3 of the Master Protocol.
Research blood sample collection on Day 1 of each cycle for patients who consent.

TABLE 2
Karnofsky Performance Status Scale
Definitions Rating (%) Criteria.

| | | |
|---|---|---|
| Able to carry on | 100 | Normal no complaints; no evidence of |
| normal activity and | | disease. |
| to work; no special | 90 | Able to carry on normal activity; minor |
| care needed. | | signs or symptoms of disease. |
| | 80 | Normal activity with effort; some signs |
| | | or symptoms of disease. |
| Unable to work; able to | 70 | Cares for self; unable to carry on |
| live at home and care for | | normal activity or to do active work. |
| most personal needs; | 60 | Requires occasional assistance but is |
| varying amount of | | able to care for most of his personal |
| assistance needed. | | needs. |
| | 50 | Requires considerable assistance and |
| | | frequent medical care. |
| Unable to care for self; | 40 | Disabled; requires special care and |
| requires equivalent of | | assistance. |
| institutional or hospital | 30 | Severely disabled; hospital admission is |
| care; disease may be | | indicated although death not imminent. |
| progressing rapidly. | 20 | Very sick; hospital admission necessary; |
| | | active supportive treatment necessary. |
| | 10 | Moribund; fatal processes progressing |
| | | rapidly. |
| | 0 | Dead. |

For more information please see Reference 17.

Schedule of Events for Troriluzole Arm. On the troriluzole arm, the evaluations defined above should occur according to the Study Calendars.

For all treatment visits (defined as including the “Required Tests or Assessments”) throughout this protocol, an administrative window of ±3 days (or longer if otherwise specified) is permitted. Treatment or visit delays for public holidays or weather conditions or force majeure do not constitute protocol violations, all efforts should be made to adhere to the stipulated assessment schedule.

End of Treatment Assessments. All the procedures described need to be completed within 14 days prior to troriluzole discontinuation or completion, or otherwise repeated for the End of Treatment Visit within 14 days of troriluzole discontinuation or completion.

All patients will be followed for safety assessment 28 days after their last dose of troriluzole.

Post-Treatment Follow-Up Assessments. Post-Treatment Follow-Up assessments should be completed as described below.

### Treatment of Patients

Patient Dosing Instructions. Subjects randomized to troriluzole experimental arm will receive the troriluzole dose that is confirmed in the dose finding phase for each cohort.

It is recommended that all subjects ingest study drug twice every day, once in the morning and once in the night (approximately 12 hours apart and at the same times each day), without regard to meals.

Newly diagnosed patients will receive TMZ at 75 mg/m² orally (7 days a week) during the treatment period, and in the maintenance period, the first cycle of TMZ will be at 150 mg/m² for Days 1-5 of a 28-day cycle. Second and subsequent cycle of maintenance therapy will be 200 mg/m² for Days 1-5 of a 28-day cycle if there are no toxicities. TMZ will be administered up to 6 cycles in the maintenance phase in combination with troriluzole. After 6 cycles, the patients will continue to receive troriluzole only.

Recurrent patients will receive lomustine at 110 mg/m² D1 of a 42-day cycle, with a maximum of 6 cycles. After 6 cycles, the patients will continue to receive troriluzole only.

Concomitant Medications. Prior and concomitant use of riluzole is prohibited. Troriluzole should be used with caution with medications that are inhibitors or inducers of the CYP1A2 enzyme system due to the potential for drug interactions, and be avoided with strong CYP1A2 inhibitors (e.g., fluvoxamine, ciprofloxacin). Subjects should be monitored appropriately when taking a CYP1A2 inhibitor or inducer. The following medications are prohibited at least 5 half-lives prior to randomization and during the study:
1. Strong to moderate CYP1A2 inhibitors which may increase the risk of riluzole associated AEs, including the SSRI fluvoxamine; strong inhibitors are prohibited.
2. Strong to moderate CYP1A2 inducers which may result in decreased efficacy.
3. Hepatotoxic drugs (e.g., allopurinol, methyldopa, sulfasalazine) which may increase the risk for hepatotoxicity.

Oral contraceptives which contain ethinyl estradiol (moderate CYP1A2 inhibitor) are allowed.

Growth Factors. For growth factors permitted for supportive care, please see below.

Anticoagulants. There are no additional guidelines for anticoagulants specific to the troriluzole arm.

Herbal Preparations/Medications. The use of medical or recreational marijuana and cannabidiol (CBD) oil is strongly discouraged 30 days prior to randomization.

Other Anti-Cancer or Experimental Therapies. There are no additional guidelines for other anti-cancer or experimental therapies specific to the troriluzole arm.

Supportive Care. There are no additional guidelines for supportive care specific to the troriluzole arm.

Antiemetic Medication. Premedication with antiemetic therapy will be permitted, if clinically indicated.

Antiepileptic Medication. There are no additional guidelines for antiepileptic medication specific to the troriluzole arm.

Dose Modifications and Toxicity Management. The following Tables 3-5 outlines the dose modifications for troriluzole. Dose modifications will be the same in the newly diagnosed and the recurrent cohort and based on the confirmed starting dose from the dose finding phase. Toxicities are graded according to National Cancer Institute [NCI] Common Terminology Criteria for Adverse Events [CTCAE] v5).

TABLE 3
Dose Modifications for Troriluzole if 100 mg BID
for two weeks with a subsequent increase to 200
mg BID is confirmed as the starting dose.

| Dose Level | Daily Troriluzole |
|---|---|
| Dose level 0 (Starting dose) | 100 mg BID for two weeks, then increased |
| | to 200 mg BID |
| Dose level −1 | 100 mg BID |

If there are clinically significant AEs that occur while the participant is on troriluzole 200 mg BID, which are judged to be related to troriluzole, then the investigator may consider modifications to the dosage regimen. Down titration to 100 mg BID (Dose level-1) is allowed after Week 2.

Troriluzole may also be held, based on the judgement of the investigator. Consultation with the medical monitor is recommended. Once the issue resolves, the dose can be titrated up to 200 mg BID as per investigator's discretion.

TABLE 4
Dose Modifications for Troriluzole if 100 mg qAM
and 200 mg qPM is confirmed as the starting dose.

| Dose Level | Daily Troriluzole |
|---|---|
| Dose level 0 (Starting dose) | 100 qAM and 200 mg qPM |
| Dose level −1 | 100 mg BID |

TABLE 5
Dose Modifications for Troriluzole if 100
mg BID is confirmed as the starting dose.

| Dose Level | Daily Troriluzole |
|---|---|
| Dose level 0 (Starting dose) | 100 mg BID |

Troriluzole should be held when there are DLTs≥Grade 3. Troriluzole can be restarted once the DLTs resolves to ≤Grade 1 at Dose level-1. The DLT should resolve within 28 days in order to restart the troriluzole. If another DLT≥Grade 3 occurs, then permanently stop the drug.

Patients who experience a toxicity described under the definition of a DLT following dose reduction will be discontinued from the study. CTCAE defines Dose-Limiting toxicity (DLT) to be a toxicity that prevents further administration of the agent at that dose level.

An AE is considered to be a DLT if it is a clinically significant AE (based on CTCAE v5.0) assessed as unrelated to tumor progression, intercurrent illness, temozolomide, radiation, or other concomitant medications.

A DLT should be defined as the following events not clearly due to the underlying disease or extraneous causes:
Any death
Non-hematologic toxicity
   Grade 3 or higher.
   Hy's law cases
Hematologic toxicity
   Grade 4 neutropenia >7 days
   Grade 3 or higher thrombocytopenia with clinically significant bleeding
   Neutropenic fever

The DLT definition may exclude:
Grade 3 nausea/vomiting or diarrhea for less than 72 hours with adequate antiemetic and other supportive care
Grade 3 fatigue for greater than 1 week.
Grade 3 or higher electrolyte abnormality that lasts up to 72 hours, is not clinically complicated, and resolves spontaneously or responds to conventional medical interventions.
Grade 3 or higher amylase or lipase that is not associated with symptoms or clinical manifestations of pancreatitis

Grade 3 or higher seizures (without further justification based on grade or baseline frequency), deep vein thrombosis and pulmonary embolism that are considered to be attributable to the study drug will not be excluded as DLTs.

Dose modifications and discontinuation of TMZ should be as described in the guidelines provided in local approved regulatory prescribing information are met. Guidelines specific for hematological toxicities (neutropenia and thrombocytopenia) should be followed as outlined in Table 6 below.

TABLE 6
TMZ and Troriluzole Dosing Interruption or Discontinuation during
Treatment and Maintenance Phase for Newly Diagnosed patients

| Toxicity | Range | Action |
|---|---|---|
| Absolute | ≥1.0 × 10⁹/L | Hold TMZ and continue |
| Neutrophil | and <1.5 × 10⁹/L | Troriluzole at Dose level 0 |
| Count (ANC) | <1.0 × 10⁹/L | Hold TMZ and Troriluzole |
| Platelet counts | ≥50 × 10⁹/L | Hold TMZ and continue |
| | and <100 × 10⁹/L | Troriluzole at Dose level 0 |
| | <50 × 10⁹/L | Hold TMZ and Troriluzole |

Dose modifications and discontinuation of lomustine should be as described in the guidelines provided in local approved regulatory prescribing information are met. Please see Table 7 below for details.

TABLE 7
Lomustine and Troriluzole Dosing Interruption or Discontinuation
during Treatment Phase for Recurrent patients

| Toxicity | Range | Action |
|---|---|---|
| Absolute | ≥1.0 × 10⁹/L | Hold Lomustine and continue |
| Neutrophil | and <1.5 × 10⁹/L | Troriluzole at Dose level 0 |
| Count (ANC) | <1.0 × 10⁹/L | Hold Lomustine and Troriluzole |
| Platelet counts | ≥50 × 10⁹/L and | Hold Lomustine and continue |
| | <100 × 10⁹/L | Troriluzole at Dose level 0 |
| | <50 × 10⁹/L | Hold Lomustine and Troriluzole |

Mild increases in liver enzymes are the most commonly observed adverse events with troriluzole. Dose modification guidelines for liver enzymes are listed in Table 8 below. In general, mild liver enzymes elevations attributed to troriluzole occur due to enzyme induction and are likely to resolve spontaneously within 12 weeks without any changes in the dose of troriluzole. However, the investigator should evaluate the subject, as appropriate.

For AST/ALT elevations greater than 3×ULN, consultation with Medical Monitor is required within 72 hours. In these cases, the clinical assessment should include repeat laboratory assessments (ALT, AST, total and direct bilirubin, alkaline phosphatase, PT, aPTT, INR) performed within 1 week and followed until resolution. Additional testing for causes of increased liver enzymes may be performed as appropriate (e.g., viral serologies). No specific management is indicated when TMZ and troriluzole are held, other than supportive care.

Guidelines for dose modifications for hepatic enzymes and bilirubin changes are listed in Table 8 below.

TABLE 8
Dose Modifications for Altered Hepatic Enzymes.

| Criteria | Dose modification |
|---|---|
| AST/ALT: 1.0-3.0 × ULN | No dose modification. |
| AND/OR | Continue TMZ and Troriluzole. |
| Bilirubin: >1.0 ULN-1.5 × ULN | |
| AST/ALT: >3.0×-5.0 × ULN | No mandatory dose modification. |
| | Continue TMZ and Troriluzole. |
| AST/ALT >3.0×-5.0 × ULN | Hold TMZ and troriluzole. |
| AND | |
| Total Bilirubin: >2 (Hy's law)* | |
| AST/ALT: >5.0 × ULN | Hold TMZ and troriluzole. |
| Bilirubin: >3.0 × ULN | |

*Unless the patient has a history or suspicion of Gilbert's syndrome.

### REFERENCES

1. Covis Pharma. Rilutek (riluzole) US Prescribing Information. Revised March 2020. www.accessdata.fda.gov/drugsatfda_docs/label/2020/020599s019lbl.pdf
2. Sanderink G J, Bournique B, Stevens J, Petry M, Martinet M. Involvement of human CYP1A isoenzymes in the metabolism and drug interactions of riluzole in vitro. J Pharmacol Exp Ther. 1997; 282 (3): 1465-1472. PMID: 9316860.
3. Dunlop J, Beal McIlvain H, She Y, Howland D S. Impaired spinal cord glutamate transport capacity and reduced sensitivity to riluzole in a transgenic superoxide dismutase mutant rat model of amyotrophic lateral sclerosis. J Neurosci. 2003; 23 (5): 1688-1696. PMID: 12629173.
4. Sung B, Lim G, Mao J. Altered expression and uptake activity of spinal glutamate transporters after nerve injury contribute to the pathogenesis of neuropathic pain in rats. J Neurosci. 2003; 23 (7): 2899-2910. PMID: 12684477.
5. Frizzo M E, Dall'Onder L P, Dalcin K B, Souza D O. Riluzole enhances glutamate uptake in rat astrocyte cultures. Cell Mol Neurobiol. 2004; 24 (1): 123-128. PMID: 15049516
6. Fumagalli E, Funicello M, Rauen T, Gobbi M, Mennini T. Riluzole enhances the activity of glutamate transporters GLAST, GLT1 and EAAC1. Eur J Pharmacol. 2008; 578 (2-3): 171-176. PMID: 18036519.
7. Banasr M, Chowdhury G M, Terwilliger R, et al. Glial pathology in an animal model of depression: reversal of stress-induced cellular, metabolic and behavioral deficits by the glutamate-modulating drug riluzole. Mol Psychiatry. 2010; 15 (5): 501-511. PMID: 18825147.
8. Chowdhury G M, Banasr M, de Graaf R A, et al. Chronic riluzole treatment increases glucose metabolism in rat prefrontal cortex and hippocampus. J Cereb Blood Flow Metab. 2008; 28 (12): 1892-1897. PMID: 18628780.
9. Niciu M J, Kelmendi B, Sanacora G. Overview of glutamatergic neurotransmission in the nervous system. Pharmacol Biochem Behav. 2012; 100 (4): 656-664. PMID: 21889952.
10. Khan A J, LaCava S, Mehta et al. The glutamate release inhibitor riluzole increases DNA damage and enhances cytotoxicity in human glioma cells, in vitro and in vivo. Oncotarget. 2019; 10 (29): 2824-2834. PMID: 31073373.
11. Sperling S, Aung T, Martin S, Rohde V, Ninkovic M. Riluzole: a potential therapeutic intervention in human brain tumor stem-like cells. Oncotarget. 2017; 8 (57): 96697-96709. PMID: 29228563.
12. Yamada, T, Tsuji, S, Nakamura, S, et al. Riluzole enhances the antitumor effects of temozolomide via suppression of MGMT expression in glioblastoma, J Neurosurg. 2020; 134 (3): 701-710. PMID: 32168477.
13. Medikonda, R., Choi, J., Pant, et al. Synergy between glutamate modulation and anti-programmed cell death protein 1 immunotherapy for glioblastoma, J Neurosurg. 2021; 13:1-10. PMID: 34388730.
14. Ashizawa T, Figueroa K P, Perlman S L, et al. Clinical characteristics of patients with spinocerebellar ataxias 1, 2, 3 and 6 in the US; a prospective observational study. Orphanet J Rare Dis 2013; 8:177. PMID: 24225362.
15. Silk A W, Saraiya B, Groisberg R, et al. A phase Ib study of troriluzole (BHV-4157) in combination with nivolumab. J Clin Oncol. 2020; 38: suppl 5; abstract 79.
16. Merck. TEMODAR® (temozolomide) US Prescribing Information. Revised: November 2019. https://www.accessdata.fda.gov/drugsatfda_docs/label/2019/021029s033lbl.pdf
17. Schag C C, Heinrich R L, Ganz P A. Karnofsky performance status revisited: reliability, validity, and guidelines. J Clin Oncol. 1984; 2 (3): 187-193. PMID: 6699671.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A method of treating glioblastoma in a patient in need of such treatment, comprising administering to the patient a first dosage form comprising a therapeutically active amount of troriluzole or a pharmaceutically acceptable salt thereof.

2. The method according to claim 1, wherein the first dosage form comprises 100 mg of troriluzole or an equivalent amount of a pharmaceutically acceptable salt thereof.

3. The method according to claim 1 or 2, wherein the first dosage form comprises 200 mg of troriluzole or an equivalent amount of a pharmaceutically acceptable salt thereof.

4. The method according to any one of claims 1 to 3, wherein the first dosage form is administered once a day.

5. The method according to any one of claims 1 to 4, wherein the first dosage form is administered twice a day.

6. The method according to any one of claims 1 to 5, wherein the duration of treatment is four weeks, and wherein troriluzole or a pharmaceutically acceptable salt thereof is administered in an amount of 100 mg twice a day for the first two weeks and in amount of 200 mg twice a day for the other two weeks.

7. The method according to any one of claims 1 to 6, wherein the total amount of troriluzole or a pharmaceutically acceptable salt thereof administered to the patient during the day is 200 mg.

8. The method according to any one of claims 1 to 7, wherein the total amount of troriluzole or a pharmaceutically acceptable salt thereof administered to the patient during the day is 300 mg.

9. The method according to any one of claims 1 to 8, wherein the total amount of troriluzole or a pharmaceutically acceptable salt thereof administered to the patient during the day is 400 mg.

10. The method according to any one of claims 1 to 9, further comprising administering to the patient a second dosage form comprising a therapeutically active amount of temozolomide or a pharmaceutically acceptable salt thereof.

11. The method according to claim 10, wherein the second dosage form comprises 75 mg of temozolomide or an equivalent amount of a pharmaceutically acceptable salt thereof.

12. The method according to claim 10 or 11, wherein the second dosage form comprises 150 mg of temozolomide or an equivalent amount of a pharmaceutically acceptable salt thereof.

13. The method according to any one of claims 10 to 12, wherein the second dosage form is administered once a day.

14. The method according to any one of claims 10 to 13, wherein the second dosage form is administered twice a day.

15. The method according to any one of claims 10 to 14, wherein the total amount of temozolomide or a pharmaceutically acceptable salt thereof administered to the patient during the day is 75 mg.

16. The method according to any one of claims 10 to 15, wherein the total amount of temozolomide or a pharmaceutically acceptable salt thereof administered to the patient during the day is 150 mg.

17. The method according to any one of claims 1 to 16, wherein the glioblastoma is newly diagnosed O⁶-methylguanine-DNA-methyltransferase (MGMT) methylated glioblastoma.

18. The method according to any one of claims 1 to 16, wherein the glioblastoma is newly diagnosed O⁶-methylguanine-DNA-methyltransferase (MGMT) unmethylated glioblastoma.

19. The method according to claim 1, further comprising administering to the patient a third dosage form comprising a therapeutically active amount of lomustine or an equivalent amount of a pharmaceutically acceptable salt thereof.

20. The method according to claim 19, wherein the third dosage form comprises 50 mg of lomustine or an equivalent amount of a pharmaceutically acceptable salt thereof.

21. The method according to claim 19 or 20, wherein the third dosage form comprises 100 mg of lomustine or an equivalent amount of a pharmaceutically acceptable salt thereof.

22. The method according to any one of claims 19 to 21, wherein the third dosage form is administered once a day.

23. The method according to any one of claims 19 to 22, wherein the third dosage form is administered twice a day.

24. The method according to any one of claims 19 to 23, wherein the total amount of lomustine or a pharmaceutically acceptable salt thereof administered to the patient during the day is 100 mg.

25. The method according to any one of claims 19 to 24, wherein the glioblastoma is recurring glioblastoma.

26. The method according to any one of claims 1 to 25, further comprising irradiating glioblastoma cells with a therapeutically effective dose of ionizing radiation.

27. A kit for use in a method of treating a patient afflicted with glioblastoma, the kit comprising:
(a) troriluzole or a pharmaceutically acceptable salt thereof;
(b) temozolomide or a pharmaceutically acceptable salt thereof; and
(b) instructions for administering troriluzole and temozolomide according to the method of any of claims 1-26.

27. A kit for use in a method of treating a patient afflicted with glioblastoma, the kit comprising:
(a) troriluzole or a pharmaceutically acceptable salt thereof;
(b) lomustine or a pharmaceutically acceptable salt thereof; and
(b) instructions for administering troriluzole and temozolomide according to the method of any of claims 1-26.
